(19) United States
(12) Patent Application Publication
(10) Pub. No.: US 2014/0030329 A1
(43) Pub. Date: Jan. 30, 2014

(54) Methods for Treating Insomnia

(71) Applicant: Chui Yu Liu, Berkeley, CA (US)

(72) Inventor: Chui Yu Liu, Berkeley, CA (US)

(21) Appl. No.: 14/111,088

(22) PCT Filed: May 10, 2011
(86) PCT No.: PCT/US11/35895

§ 371 Date: Oct. 10, 2013

(51) Int. Cl. A61K 9/28; A61K 31/554

(52) U.S. Cl. CPC A61K 9/28; A61K 31/554
USPC 424/472; 514/211.13; 540/551

(57) ABSTRACT

Methods for treating insomnia in humans not suffering from any psychiatric disorders using quetiapine.

### TECHNICAL FIELD OF THE INVENTION

The present invention relates to methods for treating insomnia in patients, not suffering from psychiatric disorders, using low dosages of quetiapine.

### BACKGROUND OF THE INVENTION

Quetiapine, also known as 2-[2-(4-]dibenzo[b,f]-[1,4]thiazepin-11-yl-1-piperazinyl)ethoxy]ethanol, is a benzothiazepine antipsychotic agent.

Quetiapine may be administered as the compound 11-[4-[2-(2-hydroxyethoxy)ethyl]-1-piperazinyl]dibenzo[b,f]-[1,4]thiazepine or as the pharmaceutically acceptable salt including the chloride, maleate, fumarate, citrate, phosphate, methane sulphonate and sulphate salts.

The preparation of quetiapine and its salts is disclosed in, for example, European Patent No. EP 240,228.

Quetiapine is currently marketed in the United States under the tradename SEROQUEL® in 25 mg, 50 mg, 100 mg, 150 mg, 200 mg, 300 mg and 400 mg dosages. At very low doses, quetiapine acts primarily as a histamine receptor blocker and α₁-adrenergic blocker. When the dose is increased, quetiapine activates the adrenergic system and binds strongly to serotonin receptors and autoreceptors. At high doses, quetiapine starts blocking significant amounts of dopamine receptors.

Quetiapine is indicated for the treatment of schizophrenia, depressive episodes associated with bipolar disorder, acute manic episodes associated with bipolar I disorder, and maintenance treatment of bipolar I disorder.

A large percentage of the adult population suffers from some form of insomnia at some point in their lives. Insomnia can occur as an occasional episode or can be chronic, which is commonly understood to involve episodes of greater than three weeks in duration. The effects of sleep deprivation resulting from insomnia are numerous and can include an inability to concentrate, a general tired feeling during the day, reduced energy levels, blurred vision, irritability and hallucinations.

Quetiapine has been used to treat insomnia in patients with comorbid psychiatric disorders as disclosed in M. E. Cates et al., Metabolic Consequences of Using Low-Dose Quetiapine for Insomnia in Psychiatric Patients, Community Ment. Health J., May 27, 2009 and Drug Use Evaluation: Low-Dose Quetiapine (Seroquel®, Seroquel XR®), Oregon State University, Drug Use Research & Management Program. Quetiapine has also been administered at high dosages to treat insomnia as disclosed in C. R. Dolder & J. McKinsey, Quetiapine for Sleep in Patients with Dementia, The Consultant Pharmacist, vol. 25, no. 10, pp. 676-79, October 2010; J. N. Wine et al., Effects of Quetiapine on Sleep in Nonpsychiatric and Psychiatric Conditions, The Annals of Pharmacotherapy, vol. 43, no. 4, pp. 707-713, Mar. 18, 2009; C. Juri et al., Quetiapine for Insomnia in Parkinson Disease: Results from an Open-Label Trial, Clinical Neuropharmacology, vol. 28, issue 4, pp. 185-87, July/August 2005; and S. Cohrs et al., Sleep-promoting Properties of Quetiapine in Healthy Subjects, Psychopharmacology, vol. 174, pp. 421-429 (2004). However, the present invention discovers for the first time that low doses of quetiapine, particularly doses of 1-20 mg and preferably 1-12.5 mg are useful for the treatment of insomnia in patients not suffering from psychiatric disorders, and in particular, elderly or geriatric patients not suffering from psychiatric disorders. As used herein the terms “elderly” and “geriatric” are used interchangeably and refer to patients of age 60 or greater and preferably of age 65 or greater.

Thus, it is an object of the present invention to provide methods for treating insomnia in patients not suffering from psychiatric disorders by administering low doses of quetiapine.

It is another object of the present invention to provide methods for treating insomnia in elderly or geriatric patients not suffering from psychiatric disorders by administering low doses of quetiapine.

It is a further object of the present invention to provide methods for treating insomnia comprising the biphasic or pulsatile administration of low doses of quetiapine to a patient not suffering from psychiatric disorders wherein the biphasic or pulsatile administration is useful for treating insomnia, and in particular, to aid in the inducement or onset of sleep and to aid in the maintenance of sleep.

These and other objects of the present invention will become apparent from a review of the appended specification.

### SUMMARY OF THE INVENTION

The present invention accomplishes the above objects and others by providing a method of treating insomnia in patients who are not suffering from a psychiatric disorder, whereby a low dosage of quetiapine or a salt thereof is administered to the patient. As used herein the terms “low dose” or “low dosage” refer to a dose range of about 1-20 mg of quetiapine based upon the free base form of quetiapine and preferably about 1-12.5 mg of quetiapine based upon the free base form of quetiapine.

Alternative embodiments of the present invention include the administration of about 3-11 mg of quetiapine or about 5-10 mg of quetiapine to patients who are not suffering from a psychiatric disorder for the purpose of treating insomnia.

The low doses of quetiapine may be administered orally in the form of an immediate release tablet or capsule. Alternatively, the low doses of quetiapine can be administered in the form of a sublingual tablet or an orally disintegrating tablet that is designed to dissolve in a patient's mouth. The low doses of quetipaine may also be in the form of a solution, suspension, syrup or powder that can be mixed with food or liquids for easier administration, especially to the elderly patients that may have a difficult time swallowing a tablet or capsule.

Another embodiment of the present invention encompasses a pulsatile dosage form comprising quetiapine or a salt thereof adapted to release according to a biphasic dissolution profile in which the first phase provides an immediate release first pulse of a therapeutically effective amount of a first low dosage of quetiapine or a salt thereof and the second phase provides a delayed second pulse of a therapeutically effective amount of a second low dosage of quetiapine or a salt thereof. The biphasic release profile of the present invention may allow the pulsatile delivery of quetiapine or a salt thereof from a single dosage form. The first phase of the pulsatile dosage form will aid in the initial onset of sleep and the second phase of the pulsatile dosage form will aid in the maintenance of sleep so that the patient should be able to obtain a full night of sleep, typically about 8 hours.

In a further embodiment of the pulsatile dosage form, the first phase should release approximately 30-70%, preferably 40-60%, of the total amount of quetiapine or a salt thereof within 30 minutes or less, preferably within 15 minutes or less, after the dosage form is administered. The second phase should release approximately 30-70%, preferably 40-60%, of the total amount of quetiapine or salt thereof approximately 2.5 to 6 hours after administration, preferably approximately 3 to 5 hours after administration.

The pulsatile dosage form of the present invention can be a capsule, tablet, multilayer tablet or multicoated tablet. The first phase, or immediate release portion, can be a single pharmaceutical unit such as an immediate release tablet or pellet. The first phase may alternatively comprise a plurality of immediate release units such as immediate release pellets, granules or mini-tablets. The first phase may also be an immediate release coating applied to a delayed release tablet, delayed release pellet, delayed release granule, or delayed release mini-tablet.

The second phase of the pulsatile dosage form may also be a single pharmaceutical unit such as a delayed release tablet or pellet. The second phase may alternatively comprise a plurality of delayed release pellets, granules or mini-tablets.

The compositions of the first phase and second phase may be combined into a tablet or capsule for administration to a patient. Once administered to a patient, the pulsatile dosage form should release all of the quetiapine or salt thereof from the first phase within 30 minutes or less, preferably within 15 minutes or less, following administration. After the release of the quetiapine or salt thereof from the first phase, there should be a period of time where substantially no quetiapine or salt thereof is released from the pulsatile dosage form. The period of time in which substantially no quetiapine or salt thereof is released following the release of the quetiapine or salt thereof from the first phase should be approximately 1 to 5 hours after the first phase has been released, preferably approximately 2 to 4 hours. Once the period of time in which substantially no quetiapine or salt thereof is released has occurred, all of the quetiapine or salt thereof contained in the second phase of the pulsatile dosage form should be released within 30 minutes or less, preferably within 15 minutes or less. As used herein the phrase “substantially no quetiapine or salt thereof is released” means 0-10%, preferably 0-5%, and most preferably 0-2% of the quetiapine or salt thereof in the second phase of the pulsatile dosage form is released.

A further embodiment of the pulsatile dosage form comprises a first phase comprising 1-10 mg, preferably 2-8 mg and most preferably 3-6 mg of quetiapine or salt thereof and a second phase comprising 1-10 mg, preferably 2-8 mg and most preferably 3-6 mg of quetiapine or salt thereof.

Dosage forms where the immediate release entity and the delayed release entity are administered simultaneously but separately are also encompassed in the present invention.

### DETAILED DESCRIPTION OF THE INVENTION

The quetiapine used in the present invention may be prepared by any known method such as those described in European Patent No. EP 240,228. The quetiapine used in the present invention may also be in the form of a pharmaceutically acceptable salt such as the chloride, maleate, fumarate, citrate, phosphate, methane sulphonate or sulphate salt of quetiapine. These salts may also be prepared according to the methods of European Patent No. EP 240,228.

The methods of treatment according to the invention typically involve administering from 1 to 20 mg, preferably 1 to 12.5 mg, and most preferably 3 to 11 mg, of quetiapine or a salt thereof to a patient suffering from insomnia but not suffering from any psychiatric disorders. The patient is preferably an elderly or geriatric patient. The methods of the present invention can be used to treat both acute and chronic insomnia. Should a salt of quetiapine be employed in accordance with the methods of the present invention, quetiapine fumarate is preferred.

The pulsatile dosage forms according to the invention typically contain from 1 to 20 mg of quetiapine or salt thereof, preferably 1 to 12.5 mg of quetiapine or salt thereof, with each phase of the pulsatile dosage form comprising 1-10 mg, preferably 2-8 mg, and most preferably 3-6 mg of quetiapine or salt thereof.

The following embodiments of the present invention are provided to be illustrative and are not intended as limiting the scope of the present invention.

One dosage form that may be used in the present invention is an immediate release or monophasic dosage form that contains from 1 to 20 mg of quetiapine or salt thereof, preferably 1 to 12.5 mg of quetiapine or salt thereof. The immediate release or monophasic dosage form may be a tablet or capsule that contains the quetiapine or salt thereof and pharmaceutically acceptable excipients such as fillers, diluents, binders, stabilizing agents, lubricants, disintegrants or mixtures thereof. These pharmaceutically acceptable excipients are well known in the art and are described in Remington, the Science and Practice of Pharmacy, 21^{st} Ed. (2006), pp. 1058-1092, published by Lippincott Williams & Wilkins; United States Pharmacopeia 27 (2004), pp. 2809-2812; and Handbook of Pharmaceutical Excipients, 5^{th} Ed. (2006), published by the Pharmaceutical Press. The dosage forms are made by methods commonly known in the art such as direct compression, wet or dry granulation, and extrusion spherionization.

Examples of acceptable fillers, sometimes referred to as diluents, include water; sugars such as lactose, dextrose, sucrose, maltose, or microcrystalline cellulose; clays; and mixtures thereof.

Binders that are useful in the present invention include pharmaceutically acceptable substances with cohesive properties. Some examples include celluloses such as hydroxypropyl methycellulose, hydroxypropyl cellulose and carboxymethycellulose sodium; polyvinylpyrrolidone; sugars; starches; and mixtures thereof.

Examples of stabilizing agents that are useful in the present invention include organic acids and alkaline metal salts of organic acids, such as succinic acid, fumaric acid, citric acid, sodium citrate, and mixtures thereof.

Examples of lubricants, glidants and/or antiadherents that may be used in the present invention include talc, magnesium stearate, calcium stearate, stearic acid, hydrogenated vegetable oils, polyethylene glycols, silicon dioxide, and mixtures thereof.

Examples of disintegrating agents that can be used in the present invention include corn starch, croscarmelose sodium, crospovidone (polyplasdone XL-10), sodium starch glycolate (EXPLOTAB® or PRIMOJEL®) or any combination of the foregoing.

Another embodiment of the immediate release or monophasic dosage form that may be used in the present invention, especially for the elderly or geriatric patients that have trouble swallowing tablets or capsules, is a rapidly disintegrating or orally disintegrating tablet. The rapidly or orally disintegrating tablets are designed to dissolve within 5 minutes or less when placed into an aqueous media such as a patient's mouth. Rapidly or orally disintegrating dosage forms are generally described in U.S. Pat. Nos. 4,371,516; 5,178,878; 5,298,261; 5,464,632; 5,587,180; 5,720,974; 5,807,576; 5,866,163; 5,869,098; 6,024,981; 6,048,541; 6,149,938 and 6,316,029.

Another form of an immediate or monophasic release dosage form that may be used in the present invention, especially for the elderly that have trouble swallowing tablets or capsules, are liquid dosage forms such as syrups, solutions or suspensions. The syrups, solutions or suspensions of the present invention typically contain pharmaceutically acceptable excipients such as a liquid carrier, i.e., water and/or alcohol, flavoring agents, stabilizing agents, coloring agents, thickening agents or mixtures thereof. The pharmaceutically acceptable excipients employed in the syrups, solutions or suspensions of the present invention are described in Remington, the Science and Practice of Pharmacy, 21^{st} Ed. (2006), pp. 745-775, published by Lippincott Williams & Wilkins; United States Pharmacopeia 27 (2004), pp. 2809-2812; and Handbook of Pharmaceutical Excipients, 5^{th} Ed. (2006), published by the Pharmaceutical Press, and further described below.

Flavoring agents that can be used in the present invention include peppermint, spearmint, wintergreen, cinnamon, coconut, coffee, chocolate, vanilla, menthol, licorice, anise, apricot, caramel, pineapple, strawberry, raspberry, grape, cherry, mixed berry, tropical fruits, mint and mixtures thereof.

Coloring agents that may be employed in the present invention include FD&C-type dyes and lakes, fruit and vegetable extracts, titanium dioxide and mixtures thereof.

Thickening agents that may be used include methylcellulose, xanthan gum, carboxymethyl cellulose, hydroxypropyl cellulose, carbomer, acacia, agar, alginate, carrageenan, gum tragacanth, collagen, carboxypolymethylene, glyceryl monostearate, monostearate, polyvinylpyrrolidone, polyacrylamide and mixtures thereof.

A further form of an immediate or monophasic dosage form that may be used in the present invention are packets or sachets that contain from 1 to 20 mg of quetiapine or salt thereof, preferably 1 to 12.5 mg of quetiapine or salt thereof and pharmaceutically acceptable excipients as previously described. The material in the individual packet or sachets are in a powder form that can be easily removed from the packet or sachet and added to food or a liquid, such as water, for administration to the patient.

Another embodiment of the present invention employs a biphasic administration that allows for the immediate release of a therapeutic amount of a low dose of quetiapine or salt thereof and a delayed release of a therapeutic amount of a low dose of quetiapine or salt thereof. The biphasic administration may comprise administering a single dosage form to the patient wherein the single dosage form allows for an immediate release of a therapeutic amount of a low dose of quetiapine or salt thereof and a delayed release of a therapeutic amount of a low dose of quetiapine or salt thereof. Alternatively, the biphasic administration may be obtained by simultaneous or concurrent administration of a dosage form that will immediately release a therapeutic amount of a low dose of quetiapine or salt thereof and a separate dosage form that will release a therapeutic amount of a low dose of quetiapine or salt thereof after a predetermined time, such as 1-5 hours after administration or when the dosage form encounters a specific pH environment of the patient's gastrointestinal tract, such as a pH greater than 5, preferably greater than 5.5, and most preferably greater than 6. The simultaneous or concurrent administration of the two separate dosage forms can occur within a few minutes of each individual administration, preferably within 5 minutes or less, and most preferably within 2 minutes or less.

One embodiment of the biphasic administration comprises the administration of a single tablet or capsule that allows for an immediate release of a therapeutic amount of a low dose of quetiapine or salt thereof, or first phase, and a delayed release of a therapeutic amount of a low dose of quetiapine or salt thereof, or second phase. The first phase, or immediate release phase, of the single tablet or capsule may be in the form of an immediate release layer or coating applied to a tablet or pellet core, an immediate release layer of a bilayer tablet, an immediate release pellet, granule, powder or mini-tablet.

The second phase, or delayed release phase, of the single tablet or capsule may be a delayed release coated tablet, pellet, granule or mini-tablet. The delayed release coated tablets, pellets, granules or mini-tablets may be made by first preparing a low dose of quetiapine or salt thereof core and coating the core with a delayed release coating. Coating methods can consist of conventional techniques commonly known in the art including spraying a solution of a polymer on the tablets, either in a pan coater or a fluid bed coating apparatus. Coating techniques are described in the literature in, for example, J. M. McGinity, Aqueous polymer coatings for Pharmaceutical Dosage Forms, Dekker N.Y. (1989) and Remington, the Science and Practice of Pharmacy, 21^{st} Ed. (2006), pp. 929-938, published by Lippincott Williams & Wilkins. Suitable delayed release coatings can be selected from enteric polymers such as zein, shellac, methacrylic acid copolymers (EUDRAGIT® S or EUDRAGIT® L), cellulose acetate phthalate, hydroxypropyl methylcellulose phthalate, hydroxypropyl methylcellulose acetate succinate, cellulose acetate trimellitate, polyvinyl acetate phthalate or mixtures thereof.

The delayed release coatings may also comprise plasticizers such as adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and combinations thereof and processing aids such as talc, magnesium stearate, calcium stearate, stearic acid, hydrogenated vegetable oils, polyethylene glycols, silicon dioxide, kaolin, and combinations thereof. Additional examples of the delayed release coating material and excipients can be found in U.S. Pat. No. 5,413,777; United Kingdom Patent No. 760,403; WO 99/03453; and EP 0475536.

The first phase, or immediate release phase, and the second phase, or delayed release phase, may be combined into a single capsule or a single tablet for administration to a patient. The single tablet may be in the form of a bilayer tablet comprising a first layer for the first phase, or immediate release phase, and a second layer for the second phase, or delayed release phase; a coated tablet comprising a delayed release core with an immediate release coating surrounding the delayed release core; or a tablet comprising delayed release pellets or granules and an immediate release component which can be in the form of a coating and particles compressed into the tablet matrix surrounding the delayed release pellets or granules.

The delayed release coating employed in various embodiments of the biphasic administration of the present invention should be applied so that the quetiapine or salt thereof allotted for the second phase is released only after the composition has passed through the stomach. To ensure that the quetiapine or salt thereof is not released until the composition has left the stomach, the delayed release coating should be designed to dissolve at a pH greater than 5.0, preferably greater than 5.5, and most preferably greater than a pH of 6.

### EXAMPLES

The following are provided by way of example only and are by no means intended to be limiting.

### Example 1

An immediate release tablet comprising 1-12.5 mg quetiapine fumarate can be prepared by mixing the following materials and compressing the mixture into a tablet.

| | | |
|---|---|---|
| quetiapine fumarate | 0.5-25 | wt % |
| filler | 25-80 | wt % |
| stabilizer | 1-25 | wt % |
| binder | 1-10 | wt % |
| lubricant | 0.2-5 | wt % |

The above tablets may optionally be coated with an enteric coating comprising an enteric polymer as previously described.

The enteric coating may be further coated with an immediate release coating containing a binder and quetiapine or salt thereof. The final tablet comprises 1-6 mg of quetiapine or salt thereof in the delayed release core and 1-6 mg of quetiapine of salt thereof in the immediate release coating.

### Example 2

A capsule comprising a mixture of immediate release quetiapine pellets and delayed release quetiapine pellets may be prepared as follows:

A suspension comprising quetiapine fumarate, sodium citrate and povidone is sprayed onto non-pariel sugar seeds in a fluid bed drier to prepare immediate release quetiapine pellets.

A portion of the immediate release quetiapine pellets are further coated with an enteric coating suspension comprising, water, acetone, hydroxypropyl propylmethyl cellulose phthalate, triethyl citrate and talc in a fluidized bed drier.

A mixture of the enteric coated pellets and immediate release quetiapine pellets are filled into a capsule so that the final capsule contains a plurality of enteric coated pellets that comprise 1-6 mg of quetiapine or salt thereof and a plurality of immediate release pellets that comprise 1-6 mg of quetiapine or salt thereof.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of treating insomnia in a human not suffering from any psychiatric disorders comprising administering to the human a dosage form comprising about 1 to about 20 mg of quetiapine or a pharmaceutically acceptable salt thereof.

2. The method of claim 1 wherein the dosage form provides for the immediate release of the quetiapine or pharmaceutically acceptable salt thereof.

3. The method of claim 2 wherein the dosage form is a tablet, capsule, orally disintegrating tablet, syrup, solution, suspension, elixir, or sachet.

4. The method of claim 1 wherein the dosage form comprises about 1 to about 12.5 mg of the quetiapine or a pharmaceutically acceptable salt thereof.

5. The method of claim 1 wherein the dosage form comprises about 3 to about 11 mg of the quetiapine or a pharmaceutically acceptable salt thereof.

6. The method of claim 1 where the human is greater than or equal to 60 years of age.

7. A method of treating insomnia in a human not suffering from any psychiatric disorders comprising administering to the human a dosage form comprising a first phase and a second phase wherein each phase comprises about 1 to about 20 mg of quetiapine or a pharmaceutically acceptable salt thereof, and wherein the first phase provides for the immediate release of the quetiapine or a pharmaceutically acceptable salt thereof and the second phase provides for the delayed release of the quetiapine or a pharmaceutically acceptable salt thereof.

8. The method of claim 7 wherein the dosage form is a bilayer tablet comprising a first layer which provides for the immediate release of the quetiapine or pharmaceutically acceptable salt thereof, and a second layer which provides for the delayed release of the quetiapine or pharmaceutically acceptable salt thereof.

9. The method of claim 7 wherein the dosage form is a coated tablet comprising a core which provides for the delayed release of the quetiapine or a pharmaceutically acceptable salt thereof wherein the core is coated with a coating that provides for the immediate release of the quetiapine or a pharmaceutically acceptable salt thereof.

10. The method of claim 7 wherein the dosage form is a tablet comprising a plurality of pellets or granules which provide for the immediate release of the quetiapine or a pharmaceutically acceptable salt thereof and a plurality of pellets or granules which provide for the delayed release of the quetiapine or a pharmaceutically acceptable salt thereof.

11. The method of claim 7 wherein all of the quetiapine or a pharmaceutically acceptable salt thereof allotted for the first phase is release within 30 minutes of the dosage form being administered to the human and all of the quetiapine or a pharmaceutically acceptable salt thereof is released at about 2.5 hours to about 6 hours after the dosage form is administered to the human.

12. The method of claim 7 wherein all of the quetiapine or a pharmaceutically acceptable salt thereof allotted for the first phase is release within 30 minutes of the dosage form being administered to the human and all of the quetiapine or a pharmaceutically acceptable salt thereof is released at about 3 hours to about 5 hours after the dosage form is administered to the human.

13. The method of claim 8 wherein each phase of the dosage form comprises about 1 to about 12.5 mg of the quetiapine or a pharmaceutically acceptable salt thereof.

14. The method of claim 8 wherein each phase of the dosage form comprises about 3 to about 6 mg of the quetiapine or a pharmaceutically acceptable salt thereof.

15. The method of claim 8 where the human is greater than or equal to 60 years of age.

16. Use of quetiapine or a pharmaceutically acceptable salt thereof in the manufacture of a medicament for treating insomnia in a human not suffering from any psychiatric disorders.
